(19) United States
(12) Patent Application Publication
(10) Pub. No.: US 2003/0108612 A1
(43) Pub. Date: Jun. 12, 2003

(54) Metallic nanoparticles for inhibition of bacterium growth

(76) Inventors: Xiaohong Nancy Xu, Norfolk, VA (US); Sophia Kyriacou, Norfolk, VA (US); Robert Jeffers, Norfolk, VA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/284,485

(22) Filed: Oct. 31, 2002

Related U.S. Application Data
(60) Provisional application No. 60/336,356, filed on Oct. 31, 2001.

(51) Int. Cl. A61K 9/14; A61K 33/24; A61K 33/38

(52) U.S. Cl. 424/489; 424/618; 424/649; 424/617

(57) ABSTRACT

Methods of inhibiting bacterial growth and treating diseases caused by bacteria by the use of metallic nanoparticles. The metallic nanoparticles have a surface comprising at least one metal and a diameter of 100 nm or less.

The present application was made under a contract from the National Institutes of Health, United States Government. The government has certain rights in this invention.

This application claims the benefit of U.S. Provisional Application No. 60/336,356, filed on Oct. 31, 2001 by Xu, Kyriacou and Jeffers, the disclosure of which is hereby incorporated by reference.

This invention is related to nanoparticles which are useful as a new class of antibiotics and can also be utilized as a new antibacterial agent in vitro. The nanoparticles are effective in inhibiting the growth of bacteria, viruses and tumors. The nanoparticles are especially useful in treating diseases caused by bacteria resistant to currently available antibiotics.

### BACKGROUND OF THE INVENTION

Despite the availability of many antibiotics having antibacterial activities, bacterial infections remain a very common health problem around the world. The problem is even more acute in underdeveloped countries. Compounded by poor and/or over crowded living conditions and the high costs of antibacterial antibiotics relative to the disposable income, bacterial infections are the most significant cause of disease-related mortality in most underdeveloped countries. There is a continuous need of antibacterial antibiotics which are easy to prepare that will be readily available to the population in underdeveloped countries.

In developed countries, although bacterial infections are not the diseases that cause the most deaths, bacterial infections still is a significant cause of mortality and morbidity. With the advances in modern medicine, a higher percentage of the population in developed countries get to live in the old age. Since the immune system deteriorates with old age, bacterial infections are a more significant health problem for this segment of the population than the younger individuals. In the old, opportunistic bacteria that are not usually pathogenic, can pose a significant health threat. Nowadays, due to a higher percentage of the population that is old than in the past, bacterial infections can become a more important public health issue even in developed countries. Similarly, opportunistic bacteria can cause diseases in individuals, such as AIDS, cancer, transplantation, bum or cystic fibrosis patients, having comprised immunity. New treatments of bacterial infections in patients that is effective in all age groups, especially the old, and in individuals even having compromised immunity will be valuable in dealing with the health threat post by bacteria, either pathogenic or opportunistic. Since the bodies of old patients and patients with compromised immunity are not as strong as that of other individuals, side effects can become a more serious problem in these patients. The new treatments of bacterial infections should be low in toxicity in order to avoid any significant complications due to side effects of the antibiotics.

In both underdeveloped and developed countries, with the increasing use of antibacterial antibiotics, the incidence of antibiotic-resistant infections has been on the rise. The fact that more and more bacterial diseases are caused by bacteria that are resistant to currently available antibiotics is another reason why new antibacterial antibiotics, especially substances that do not belong to any of the currently available classes of antibiotics, are needed.

The present inventions addresses the above needs of antibiotics by providing metallic nanoparticles as a new class of antibiotics that are useful in inhibiting the growth of bacteria. The new class of antibiotics can be made relatively easily, has a wide spectrum of antibacterial activities, causes few side effects and is effective against bacteria that are resistant to currently available antibiotics. The new class of antibiotics are also useful in inhibiting the growth of viruses, and even tumors. Additionally, the metallic nanoparticles can be utilized as antibacterial agents in vitro.

### SUMMARY OF THE INVENTION

One of the objects of the invention is directed to a method of treating a disease caused by a bacteria in a subject in need of such treatment, comprising administering a bacterial disease treating effective amount of metallic nanoparticles to the subject, wherein the metallic nanoparticles have a surface comprising at least one metal and are about 100 nm or less in diameter. Preferably, in this method of the invention, the at least one metal on the surface of the metallic nanoparticles is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

Another object of the invention is related to an in vitro use of the metallic nanoparticles. The in vitro use is directed to a method of inhibiting bacterial growth in a liquid sample, comprising contacting a liquid sample with a bacterial growth inhibition effective amount of metallic nanoparticles to inhibit the growth of bacteria in the liquid sample, wherein the metallic nanoparticles have a surface comprising at least one metal and are 100 nm or less in diameter. In this in vitro method of the invention, preferably, the at least one metal on the surface of the metallic nanoparticles is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc. The in vitro method is useful in in vitro diagnostic tests involving a liquid sample, wherein bacterial growth in the liquid sample may interfere with the diagnostic tests. The in vitro method is also useful in the culturing of cells, wherein the liquid sample can be a cell culture medium, in which maintaining a sterile condition is important.

A further subject of the invention is an in vitro or in vivo method of inhibiting the growth of a bacteria, comprising contacting the bacteria with a bacterial growth inhibition effective amount of metallic nanoparticles, wherein the metallic nanoparticles have a surface comprising at least one metal and are about 100 nm or less in diameter. In this method of the invention, the at least one metal on the surface preferably is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc. The bacterial growth inhibition effective amount of the metallic nanoparticles is preferably about 10 pM or more.

Also within the scope of the invention is a method of killing a bacterial cell, comprising contacting a surface of the bacterial cell with a bacterial cell killing effective amount of metallic nanoparticles, wherein the metallic nanoparticles have a surface comprising at least one metal and are about 100 nm or less in diameter. In this method of the invention, the at least one metal on the surface preferably is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc. The bacterial cell killing effective amount of the metallic nanoparticles is preferably about 3 or more metallic nanoparticles per bacterial cell.

### BRIEF DESCRIPTION OF THE DRAWINGS

FIG. 1 shows the absorbances at 600 nm of different culture media containing three strains of P. aeruginosa cells exposed to silver coated gold particles at different concentrations. “Silver PRPs” stands for silver coated gold particles, the metallic nanoparticles in this example.

FIG. 2 shows the total number of P. aeruginosa cells in the absence or presence of silver coated gold particles in 5 pictures as determined by dark-field microscopy.

FIGS. 3a and 3b show the total number of P. aeruginosa cells in the presence of different concentrations of azthreonam (AZT) or gentamicin in 5 pictures as determined by dark-field microscopy.

### DETAILED DESCRIPTION OF THE INVENTION

In the methods according to the invention, the metallic nonoparticles comprise at least one metal on the surface, wherein the at least one metal more preferably is selected from the group consisting of gold, silver, platinum and palladium. Even more preferably, the at least one metal on the suface is gold or silver. The at least one metal, most preferably, is silver.

The metallic nanoparticles used in the invention have a surface and a core. The surface comprises at least one metal, while the core can be made of any solid material, e.g. at least one metal or polymer. Examples of the at least one metal forming the core are gold, silver, platinum, osmium, iridium, ruthenium, rhodium, palladium, aluminum, chromium, cobalt, copper, iron, magnesium, nickel, tantalum, tin, titanium, tungsten, vanadium, zinc or mixtures thereof. Preferably, the at least one metal forming the core is gold, silver, platinum or palladium. More preferably, the at least one metal forming the core is gold or silver. The at least one metal forming the core most preferably is gold. Examples of polymeric materials used to form the core of the metallic nanoparticles are polystyrene, polyethylene, polypropylene, polycarbonate and polyurethane, with polystyrene being preferred.

When the core of the metallic nanoparticles is formed of the at least one metal, the at least one metal of the core and the at least one metal on the surface are the same or different. In some of the embodiments of the invention, the at least one metal on the surface is different from the at least one metal of the core of the metallic nanoparticles. For example, metallic nanoparticles having silver on the surface and a metal selected from gold, platinum and palladium in the core can be used in any of the methods according to the invention. Preferably, the metallic nanoparticles have silver on the surface and gold in the core.

The surface of the metallic nanoparticles used in the invention comprises at least one metal. Within the scope of the invention are the use of nanoparticles, wherein the complete surface of the metallic nanoparticles is formed entirely of the at least one metal, e.g. gold, silver, platinum, osmium, iridium, ruthenium, rhodium, palladium, aluminum, chromium, cobalt, copper, iron, magnesium, nickel, tantalum, tin, titanium, tungsten, vanadium, zinc or mixtures thereof. Preferably, the complete surface of the metallic nanoparticles is formed entirely of gold, silver, platinum, palladium or mixtures thereof. The complete surface of the metallic nanoparticles used in the invention is formed entirely of, more preferably, gold or silver and most preferably silver.

The metallic nanoparticles used in the methods of the invention have a diameter of 100 nm or less. Preferably, the metallic nanoparticles have a diameter between about 30 nm and about 100 nm, and more preferably, between about 50 nm and about 100 nm. The metallic nanoparticles have a diameter, even more preferably, between about 60 nm and about 80 nm. Also within the scope of the invention is the use of metallic nanoparticles, wherein the diameter of the metallic nanoparticles is less than about 80 nm, preferably less than about 60 nm or less than about 40 nm.

The present invention is based on a discovery that the metallic nanoparticles have antibacterial activities. The bacteria sensitive to the metallic nanoparticles include gram positive bacteria, gram negative bacteria, spirochetes e.g. Treponema pallidum, Treponema pertenue, Treponema carateum, Leptospira interrogans, Borrelia hermsii, Borrelia turicatae, Borrelia parkeri, and Borrelia burgdorferi, and acid fast bacteria, e.g. Mycobacterium tuberculosis, Mycobacterium bovis, Mycobacterium africanum, Mycobacterium microti and Mycobacterium leprae. Examples of gram positive bacteria sensitive to the metallic nanoparticles include Staphylococcus aureus, Staphylococcus epidermidis, Streptococcus pyogenes, Streptococcus pneumoniae, Streptococcus agalactiae, Enterococcus faecalis, Enterococcus faecium, Enterococcus bovis, Corynebacterium diphtheriae, Listeria monocytogenes, Bacillus anthracis, Clostridium perfringens, Clostridium difficile, Clostridium botulinum, Clostridium tetanus, and Clostridium novyi. Examples of gram negative bacteria sensitive to the metallic nanoparticles include Pseudomonas aeroginosa, Neisseria gonorrhoeae, Neisseria meningitidis, Haemophilus influenzae, Haemophilus parainfluenza, Haemophilus haemolyticus, Haemophilus parahaemolyticus, Haemophilus aphrophilus, Klebsiella pneumoniae, Campylobacter fetus, Campylobacter jejuni, Campylobacter coli, Helicobacter pylori, Vibrio cholerae, Vibrio mimicus, Salmonella typhimurium, Salmonella enteritidis, Shigella sonnei, Shigella boydii, Shigella flexneri, Shigella dysenteriae, Escherichia coli, Brucella melitensis, Brucella abortus, Brucella suis, Rickettsia rickettsii, Francisella tularensis, Pasteurella multocida, Yersinia pestis, Yersinia enterocolitica, Yersinia pseudotuberculosis, Proteus mirabilis, Bacteroides spp., Fusobacterium spp., Bordetella pertussis, and Legionella pneumophila.

The present invention is useful in treating a disease caused by a bacteria comprising administering an effective amount of the metallic nanoparticles to a subject in need of such a treatment, wherein the bacteria is sensitive to the metallic nanoparticles. The method is particularly useful when the bacteria is resistant to an antibiotic other than the metallic nanoparticles. In addition to the administration of the metallic nanoparticles, an antibiotic other than the metallic nanoparticles can be administered to the subject. Examples of such other antibiotics include penicillins and related drugs, carbapenems, cephalosporins and related drugs, aminoglycosides, bacitracin, gramicidin, mupirocin, chloramphenicol, thiamphenicol, fusidate sodium, lincomycin, clindamycin, macrolides such as erythromycin, roxithromycin and clarithromycin, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, imipenen, amikacin, netilmicin, fosfomycin, gentamicin, ceftriaxone and teicoplanin.

The subject that is treated in the antibacterial methods of the present invention is an animal. Preferably, the subject is a mammal. More preferably, the subject is a human, such as a child or an adult.

In the method of the present invention for treating a bacterial disease or inhibiting the growth of bacteria, the metallic nanoparticles can be administered to the subject being treated at a dose of 0.01 mg/kg body weight to 10 g/kg body weight. Preferably, the dose is 0.1 mg/kg to 1 g/kg. More preferably, the dose is 1 mg/kg to 100 mg/kg. The doses disclosed above can be adjusted by one skilled in the art based on the severity of the disease, the virulence of the bacteria, the age, sex and health condition of the subject.

In the method of the present invention for treating a bacterial disease or inhibiting the growth of bacteria, the metallic nanoparticles can be administered topically, orally or parentally. Preferably, the metallic nanoparticles are administered orally or intravenously. When the metallic nanoparticles are administered orally, the method is particularly useful in treating gastrointestinal infection by the bacteria. When the metallic nanoparticles are administered intravenously, the method is particularly effective in treating bacteremia or bacterial infections of internal organs.

An object of the invention is also directed to the methods of using the metallic nanoparticles in vitro. These in vitro methods take advantage of the antibacterial activities of the metallic nanoparticles. By inhibiting or preventing the growth of bacteria, the in vitro methods of the invention are useful in clinical diagnostic tests or in cell culturing applications, in which bacterial growth is undesirable.

In one of the in vitro methods of the invention, bacterial growth in a liquid sample is inhibited or prevented by contacting the liquid sample with a bacterial growth inhibition effective amount of the metallic nanoparticles in order to inhibit the growth of a bacteria. For instance, an antibacterial effective amount of the metallic nanoparticles can be mixed with the liquid sample to inhibit or prevent bacterial growth in the liquid sample.

In another method of the invention, which may be in vitro or in vivo, bacterial growth is inhibited by contacting the surface of the bacteria to a bacterial growth inhibition effective amount of the metallic nanoparticles.

In both in vitro and in vivo uses, the bacterial growth inhibition effective amount of the metallic nanoparticles can be a concentration of about 10 pM or more. Preferably, the bacterial growth inhibition effective amount of the metallic particles is a concentration of about 20 pM or more, more preferably about 40 pM or more, and most preferably about 80 pM or more. For the methods described above, the liquid sample can be a sample of blood, serum, plasma, saliva, cerebral spinal fluid, urine, and cell culture medium.

Another method according to the invention, which may be in vitro or in vivo, is a method of killing a bacterial cell, comprising contacting a surface of the bacterial cell with a bacterial cell killing effective amount of metallic nanoparticles. In this method, the bacterial cell killing effective amount can be about 3 or more metallic nanoparticles contacting the bacterial surface per bacterial cell. Preferably, the bacterial cell killing effective amount is about 3 to about 10, more preferably about 3 to about 6, metallic nanoparticles contacting the bacterial surface per bacterial cell.

The metallic nanoparticles used in the methods of the invention can be prepared by methods known in the art. For instance, silver is deposited on metal particles having a diameter of less than 100 nm using commercially available silver enhancement kits, e.g. see Schultz et al., Proc. Nat'l Acad. Sci., USA, 2000, 97:996.

The invention is demonstrated by working examples shown below. The examples are for illustration purposes only, and should not be used to limit the scope of the invention. The scope of the invention is measured by the claims, not the examples. Any modification, by a person skilled in the art, of the examples based on the claims using the disclosures herein and a knowledge of the art is within the scope of the invention.

### EXAMPLE 1

Preparation of Metallic Nanoparticles

Metallic nanoparticles having a silver surface and gold core were prepared. Gold particles having a diameter of 6.5 nm were first prepared as described herein. Glassware was cleaned with royal water (a mixture of hydrochloric acid to nitric acid, 1:3), then rinsed with nanopore-filtered water, and then dried prior to use. An aqueous solution of HAuCl₄ (1 mM, 500 mL) was brought to a reflux while stirring, and then 10 mL of 1% (w/v) tri-sodium citrate and 2.25 mL of 1% tannic acid were added quickly, which resulted in a color change from yellow to deep red. After the color change, the solution was refluxed for another 5 minutes and allowed to cool to room temperature, and subsequently filtered through a 0.22 μm filter to obtain a colloid of gold particles. The gold particles were characterized by transmission electron microscopy to have a size of 6.5 nm and a spherical shape. This colloid of 6.5 nm gold particles was characterized by UV-vis spectroscopy to have an absorbance of 0.7742 at 520 nm.

The metallic nanoparticles were prepared by enhancing 6.5 nm colloidal gold nucleating cores until a desired size of particles was achieved using a commercially available silver enhancement kit. Three populations of silver coated gold particles (the metallic nanoparticles in this example) having a plasmon resonance peak wavelength at 458-nm (blue), 539-nm (green) or 663-nm (red) were prepared by adding 100 μL of an initiator from the silver enhancement kit into 20 mL ultra-pure water containing 6.5 nm gold particles at 5.2 pM (5.2×10⁻¹²M), followed by addition of 10 μL, 30 μL, and 60 μL of a silver enhancer into the reaction mixtures, respectively. The reaction mixtures were continuously stirred at room temperature during the entire process. The reaction was completed after about 2 min. These three populations of metallic nanoparticles were then characterized by TEM, UV-vis spectroscopy and dark-field microscopy. The spectra of the individual metallic nanoparticles demonstrated that the plasmon resonance spectra of these metallic nanoparticles showed size dependence: blue at 458 nm, green at 539 nm, and red at 663 nm for single metallic nanoparticles at a diameter of 52 nm, 74 nm and 97 nm, respectively.

### EXAMPLE 2

Inhibition of Growth and Division of Bacterial Cells

Three strains of Pseudomonas aeruginosa cells (WT, na1B, ΔABM) were used in this working example. “WT” stands for wild type P. aeruginosa, PA04290. The symbol “AABM” stands for a mutant of wild type P. aeruginosa, PA04290, lacking MexA, MaxB and OprM, wherein MexA, MexB and OprM are component genes of an efflux system in wild type P. aeruginosa. The symbol “na1B” stands for a mutant of wild type P. aeruginosa, PA04290, having over expression of the MexA, MexB and OprM genes. These three strains were precultured in autoclaved test tubes overnight. Each test tube contained 3 mL of L-broth medium (1% tryptone, 0.5% yeast extract and 0.5% NaCl, pH 7.2) and was placed inside a shaker at 37° C. and 230 rpm. Then, 20 μL of this pre-cultured cell medium was transferred to each new test tube containing 2 mL of L-broth medium and 0, 2, 20, 40, 80, 100, 200 and 400 μL of a sterile liquid containing 300 pM silver enhanced gold particles (as the metallic nanoparticles used in this example) prepared as described in Example 1. The final volume of the medium was adjusted to 3 mL using autoclaved ultra-pure water that was used to prepare the metallic nanoparticles. Thus, each test tube contained 2 mL of medium and 1 mL of ultra-pure water with final metallic nanoparticle concentrations at 0, 0.2, 2, 4, 8, 10, 20 and 40 pM, respectively. These test tubes were then placed in the shaker at 37° C. with shaking at 230 rpm overnight. Based on visual inspection of test tubes (the presence or absence of turbidity in the medium in each of the test tube) after the overnight incubation, it was determined that the bacterial cells grew at a concentration of the silver coated gold particles of less than 20 pM, but not at a concentration of 20 pM or more. The minimal inhibitory concentration, MIC, of the silver coated gold particles (the metallic nanoparticles in this example) was about 20 pM in P. aeruginosa.

The MIC of the silver coated gold particles tested was also determined, as described below, using both UV-vis spectroscopy and single-cell dark-field microscopy, wherein P. aeruginosa cells were grown overnight at 37° C., with shaking at 230 rpm, in the presence of silver coated gold particles in L-broth medium similar to the procedures for the visual inspection experiment described above, except that final concentrations of 0, 0.2, 2, 20 and 90 pM silver coated gold particles (prepared as described in Example 1) were used.

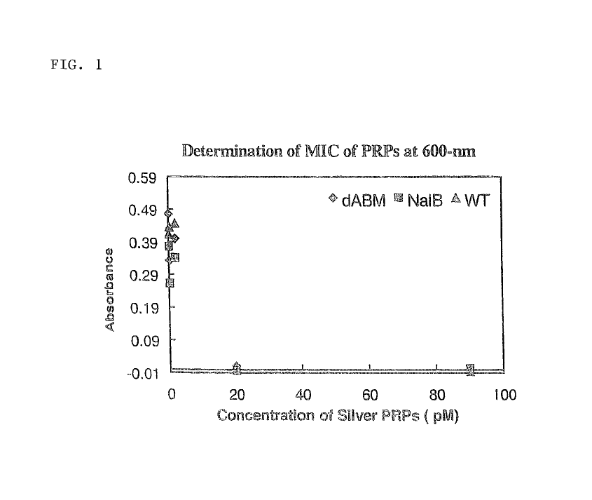

After the overnight incubation, the absorbance of the culture medium containing P. aeruginosa cells in the presence of silver coated gold particles at 0, 0.2, 2, 20 or 90 pM were measured at 600 nm using an UV-vis spectrometer to determine the concentrations of bacterial cells based upon the light scattering of bacterial cells for the three strains of P. aeruginosa, WT, na1B and ΔABM. For each strain of P. aeruginosa cells and each concentration of the silver coated gold particles, the absorbances of 5 samples of the culture medium were measured with the absorbance of each sample measured 3 times and the average absorbance was calculated. The absorbance of ultra-pure water containing the corresponding concentration of silver coated gold particles was subtracted from the average absorbance and the resulting absorbance was plotted in FIG. 1 versus the concentrations of the silver coated gold particles used in the incubation. For each of the three strains of P. aeruginosa, at a metallic nanoparticle concentration of 20 or 90 pM, the absorbance was almost zero indicating essentially no bacterial cell growth, while at a metallic nanoparticle concentration of 0, 0.2 or 2 pM the absorbance was substantially higher than zero. Thus, the MICs of the silver coated gold particles were determined to be about 20 pM for these three strains of P. aeruginosa.

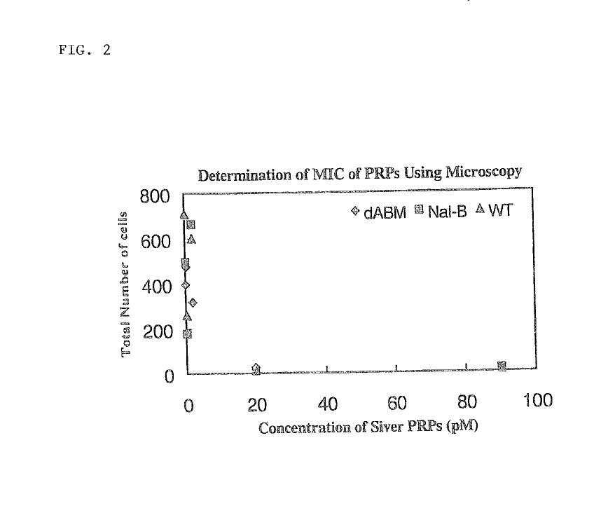

For each strain of P. aeruginosa cells and each concentration of the silver coated gold particles, the concentrations of P. aeruginosa cells in 5 samples of the culture medium were also estimated after the overnight incubation using dark-field microscopy. Images were taken of the 5 samples using a dark-field microscope equipped with a Micromax CCD camera. For each of the three strains of P. aeruginosa, 5 samples of the culture medium containing P. aeruginosa cells in the presence of silver coated gold particles at a concentration of 0, 0.2, 2, 20 or 90 pM were examined using dark-field microscopy to count the number of P. aeruginosa cells present. For each sample, 5 pictures were taken at different locations to obtain representative numbers of P. aeruginosa cells present in each sample and the numbers of P. aeruginosa cells counted in the 5 pictures were summed to obtain the total number of P. aeruginosa cells in the 5 pictures. FIG. 2 plots the total number of P. aeruginosa cells from these 5 pictures versus a given concentration of the silver coated gold particles in the culture medium. FIG. 2 demonstrates that, at a metallic nanoparticle concentration of 20 or 90 pM, there were hardly any P. aeruginosa cells in 5 pictures of the culture medium. However, at a metallic nanoparticle concentration of 0, 0.2 or 2 pM, there were 200 or more P. aeruginosa cells in the 5 pictures of the culture medium. Thus, the MICs of the silver coated gold particles were determined to be about 20 pM for the three strains of P. aeruginosa. The dark-field microscopy also demonstrated that the silver coated gold particles were either inside the P. aeruginosa cells or attached onto the P. aeruginosa cells' surface. There were green, blue and red metallic nanoparticles, but the most abundant were green. The red nanoparticles appeared to be the brightest under the dark-field microscope because of the larger size.

### EXAMPLE 3

Comparative Example Using Prior Art Antibiotics

For comparison purposes, a study was performed with procedures similar to the procedures of Example 2. Three strains, WT, na1B and ΔABM, of P. aeruginosa were used. Instead of the metallic nanoparticles, azthreonam or gentamicin was used as the antibiotic. From the published literature, it was known that azthreonam and gentamicin both have a MIC of about 3.13 μg/mL for WT P. aeruginosa.

The three strains of P. aeruginosa cells were pre-cultured in autoclaved, test tubes overnight. Each test tube contained 3 mL of L-broth medium (1% tryptone, 0.5% yeast extract and 0.5% NaCl; pH 7.2) and was placed inside a shaker at 37° C. and 230 rpm. Then, 20 μL of this pre-cultured cell medium was transferred from each of the test tube to separate new test tubes containing 2 mL of L-broth medium and the antibiotics, azthreonam or gentamicin. The final volume of the sample in each of the new test tube was adjusted to 3 mL by adding 1 mL of autoclaved ultra-pure water that was used to prepare the antibiotics to achieve a final antibiotic concentration of 0, 1/10 the known MIC, the known MIC, 5× the known MIC or 10× the known MIC, wherein 3.13 μg/mL was the known MIC. Thus, each of the new test tube contained 2 mL of pre-cultured cell medium, 1 mL of ultra-pure water and azthreonam or gentamicin at a final antibiotic concentration of 0, 0.313, 3.13, 15.65 or 31.3 μg/mL. These test tubes were then placed in the shaker at 37° C. and 230 rpm overnight. Visual inspections of the samples after the overnight incubation demonstrated that the P. aeruginosa cells grew at antibiotic concentrations less than 3.13 μg/mL, but not at 3.13 μg/mL or higher, indicating that azthreonam and gentamicin indeed inhibited P. aeruginosa cell growth at their minimum inhibitory concentrations (MICs) of about 3.13 μg/mL.

Using UV-vis spectroscopy, the MIC for AZT or gentamicin was also determined to be about 3.13 μg/mL. With a UV-vis spectrometer, the absorbance of each sample of the culture medium was measured at 600 nm after the overnight incubation to determine the P. aeruginosa cell concentration based upon light scattering of cells. For each strain of P. aeruginosa cells, the absorbances of 4 samples of the P. aeruginosa culture medium containing azthreonam or gentamicin at a concentration of 0.313, 3.13, 15.65 or 31.3 μg/mL were measured. The absorbance of each of the 4 samples was measured 3 times and the average absorbance was calculated. Then, the absorbance of ultra-pure water used to prepare the antibiotic was subtracted from the average absorbance. For the three strains of P. aeruginosa growing in azthreonam or gentamicin at a concentration of 0.313 g/mL, the absorbances at 600 nm were about 0.08 or higher. In contrast, for the three strains of P. aeruginosa in azthreonam or gentamicin at a concentration of 3.13, 15.65 or 31.3 μg/mL, the absorbances at 600 nm were almost zero indicating no P. aeruginosa cell growth at the MIC of 3.13 μg/mL.

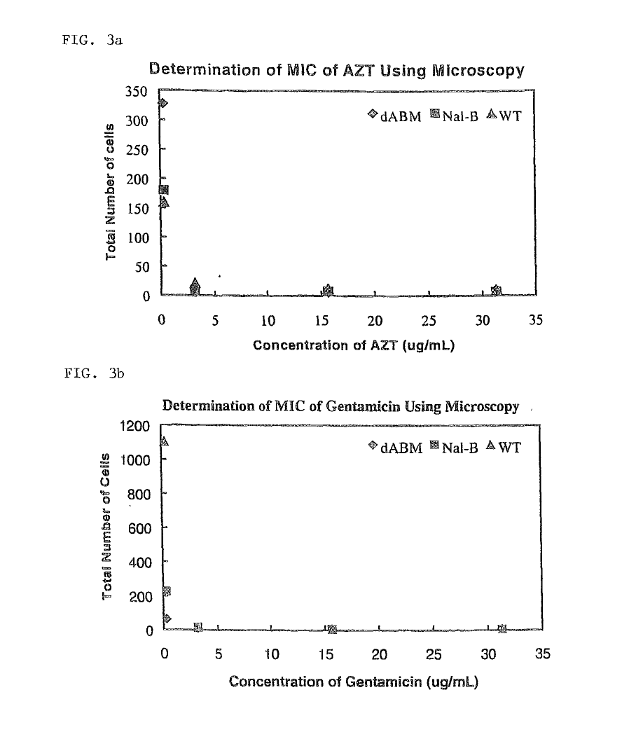

Similarly, with single-cell dark-field microscopy, the MIC for azthreonam or gentamicin was determined to be about 3.13 μg/mL. After the overnight incubation described above, images were taken using a dark-field microscope equipped with a Micromax CCD camera. For each of the three strains of P. aeruginosa in the presence of azthreonam or gentamicin at a concentration of 0.313, 3.13, 15.65 or 31.3 μg/mL, 4 samples of the culture medium were subjected to single-cell dark-field microscopy. For each of the sample, 5 pictures were taken at different locations using the dark-field microscope with the camera to obtain representative numbers of P. aeruginosa cells present in the medium sample and the number of P. aeruginosa cells in the 5 pictures were summed to obtain the total number of P. aeruginosa cells in the 5 pictures. At the azthreonam or gentamicin concentration of 3.13, 15.65 or 31.3 μg/mL, the total number of P. aeruginosa cells present in the 5 pictures was almost zero indicating that the MICs of azthreonam and gentamicin for the three strains of P. aeruginosa were indeed about 3.13 μg/mL (see FIG. 3)

The MICs determined using these methods were consistent with the results reported by the literature for azthreonam and gentamicin. These experiments described above demonstrated that the MIC of the silver coated gold particles (the metallic nanoparticles used in Example 2) for P. aeruginosa was about 1000 times lower than the MICs for two prior art antibiotics, azthreonam and gentamicin.

### EXAMPLE 4

Heterogeneous Distribution of Number of Nanoparticles Within Individual Cells

P. aeruginosa cells of the na1B strain growing in L-broth medium containing 1% tryptone, 0.5% yeast extract and 0.5% sodium chloride, pH 7.2, were mixed with silver coated gold particles prepared as described in Example 1 at a final concentration of 5.19 pM and the medium mixture was incubated for 3.5 hours in a shaker at 37° C. with a speed of 230 rpm. After the incubation, images were taken of the medium mixture containing the bacteria using dark-field microscopy with a dark-field microscope (Nikon-E600) equipped with an oil dark-field condenser, a 100×objective lens, a PID 1030×1300 pixel CCD camera (Roper Scientific, Micromax, 5 Mhz Interline, pixel size at 0.067 μm via the 100×objective lens) for high resolution cell imaging. A heterogeneous distribution of the number of metallic nanoparticles within individual P. aeruginosa cells was observed. The majority of the P. aeruginosa cells did not have the metallic nanoparticles either inside or in contact with the cell surface. About 10% of the P. aeruginosa cells had 1 to 2 metallic nanoparticles, and approximately 1% of the P. aeruginosa cells had 3 to 6 metallic nanoparticles. P. aeruginosa cells having 3 to 6 nanoparticles on the cell surface were subject to nanoparticle aggregation, which led to cell death. With the invention not being bound by any theory on the mechanism of nanoparticle aggregation, it appeared that the 3 to 6 nanoparticles contacting the cell surface might have lost their colloidal nature and served as points of nucleation for growth of other metallic nanoparticles. Alternatively, the increasing number of nanoparticles on the cell surface might have made it impossible for the nanoparticles to remain sufficiently separated, and hence they tended to aggregate together.

What is claimed is:

1. A method of inhibiting bacterial growth in a liquid sample, comprising contacting a liquid sample with a bacterial growth inhibiting effective amount of metallic nanoparticles to inhibit the growth of bacteria in the liquid sample, wherein the metallic nanoparticles have a surface comprising at least one metal and are 100 nm or less in diameter.

2. The method of claim 1, wherein the at least one metal on the surface is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

3. The method of claim 2, wherein the at least one metal on the surface is selected from the group consisting of gold, silver, platinum and palladium.

4. The method of claim 3, wherein the at least one metal on the surface is gold or silver.

5. The method of claim 4, wherein the at least one metal on the surface is silver.

6. The method of claim 5, wherein the surface of the metallic nanoparticles comprises silver and gold.

7. The method of claim 1, wherein the metallic nanoparticles comprise the at least one metal on the surface and at least one metal in the core, wherein the at least one metal on the surface and the at least one metal in the core are the same or different.

8. The method of claim 7, wherein the at least one metal on the surface and the at least one metal in the core are independently selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

9. The method of claim 8, wherein the at least one metal on the surface and the at least one metal in the core are independently selected from the group consisting of gold, silver, platinum and palladium.

10. The method of claim 9, wherein the at least one metal on the surface and the at least one metal in the core are independently gold or silver.

11. The method of claim 7, wherein the at least one metal on the surface and the at least one metal in the core are different.

12. The method of claim 1 1, wherein the at least one metal on the surface is silver and the at least one metal in the core is gold.

13. The method of claim 1, wherein the metallic nanoparticles are of a diameter between about 50 nm and about 100 nm.

14. The method of claim 1, wherein the liquid sample is selected from the group consisting of urine, blood, serum, plasma, cerebral spinal fluid and saliva.

15. The method of claim 1, wherein the liquid sample contains a gram positive bacteria.

16. The method of claim 1, wherein the liquid sample contains a gram negative bacteria.

17. A method of treating a disease caused by a bacteria in a subject in need of such treatment, comprising administering a bacterial disease treating effective amount of metallic nanoparticles to the subject, wherein the metallic nanoparticles have a surface comprising at least one metal and are about 100 nm or less in diameter.

18. The method of claim 17, wherein the at least one metal on the surface is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

19. The method of claim 18, wherein the at least one metal on the surface is selected from the group consisting of gold, silver, platinum and palladium.

20. The method of claim 19, wherein the at least one metal on the surface is gold or silver.

21. The method of claim 20, wherein the at least one metal on the surface is silver.

22. The method of claim 21, wherein the surface of the metallic nanoparticles comprises silver and gold.

23. The method of claim 17, wherein the metallic nanoparticles comprise the at least one metal on the surface and at least one metal in the core, wherein the at least one metal on the surface and the at least one metal in the core are the same or different.

24. The method of claim 23, wherein the at least one metal on the surface and the at least one metal in the core are independently selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

25. The method of claim 24, wherein the at least one metal on the surface and the at least one metal in the core are independently selected from the group consisting of gold, silver, platinum and palladium.

26. The method of claim 25, wherein the at least one metal on the surface and the at least one metal in the core are independently gold or silver.

27. The method of claim 23, wherein the at least one metal on the surface and the at least one metal in the core are different.

28. The method of claim 27, wherein the at least one metal on the surface is silver and the at least one metal in the core is gold.

29. The method of claim 17, wherein the metallic nanoparticles are of a diameter between about 50 nm and about 100 nm.

30. The method of claim 17, wherein the bacteria is a gram positive bacteria.

31. The method of claim 30, wherein the gram positive bacteria is selected from the group consisting of Staphylococcus aureus, Staphylococcus epidermidis, Streptococcus pyogenes, Streptococcus pneumoniae, Streptococcus agalactiae, Enterococcus faecalis, Enterococcus faecium, Enterococcus bovis, Corynebacterium diphtheriae, Listeria monocytogenes, Bacillus anthracis, Clostridium perfringens, Clostridium difficile, Clostridium botulinum, Clostridium tetanus, and Clostridium novyi.

32. The method of claim 17, wherein the bacteria is a gram negative bacteria.

33. The method of claim 32, wherein the gram negative bacteria is selected from the group consisting of Pseudomonas aeroginosa, Neisseria gonorrhoeae, Neisseria meningitidis, Haemophilus influenzae, Haemophilus parainfluenza, Haemophilus haemolyticus, Haemophilus parahaemolyticus, Haemophilus aphrophilus, Klebsiella pneumoniae, Campylobacter fetus, Campylobacter jejuni, Campylobacter coli, Helicobacter pylori, Vibrio cholerae, Vibrio mimicus, Salmonella typhimurium, Salmonella enteritidis, Shigella sonnei, Shigella boydii, Shigella flexneri, Shigella dysenteriae, Escherichia coli, Brucella melitensis, Brucella abortus, Brucella suis, Rickettsia rickettsii, Francisella tularensis, Pasteurella multocida, Yersinia pestis, Yersinia enterocolitica, Yersinia pseudotuberculosis, Proteus mirabilis, Bacteroides spp., Fusobacterium spp., Bordetella pertussis, and Legionella pneumophila.

34. The method of claim 17, wherein the bacteria is selected from the group consisting of Treponema pallidum, Treponema pertenue, Treponema carateum, Leptospira interrogans, Borrelia hermsii, Borrelia turicatae, Borrelia parkeri, Borrelia burgdorferi, Mycobacterium tuberculosis, Mycobacterium bovis, Mycobacterium africanum, Mycobacterium microti and Mycobacterium leprae.

35. The method of claim 17, wherein the bacteria is resistant to antibiotics other than the metallic nanoparticles.

36. The method of claim 17, further comprising administering an antibiotic other than the metallic nanoparticles to the subject.

37. The method of claim 17, wherein the subject is a mammal.

38. The method of claim 37, wherein the subject is a human.

39. A method of inhibiting the growth of a bacteria, comprising contacting the bacteria with a bacterial growth inhibition effective amount of metallic nanoparticles, wherein the metallic nanoparticles have a surface comprising at least one metal and are about 100 nm or less in diameter.

40. The method of claim 39, wherein the at least one metal on the surface is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

41. The method of claim 40, wherein the at least one metal on the surface is selected from the group consisting of gold, silver, platinum and palladium.

42. The method of claim 41, wherein the at least one metal on the surface is gold or silver.

43. The method of claim 42, wherein the at least one metal on the surface is silver.

44. The method of claim 43, wherein the surface of the metallic nanoparticles comprises silver and gold.

45. The method of claim 39, wherein the metallic nanoparticles comprise the at least one metal on the surface and at least one metal in the core, wherein the at least one metal on the surface and the at least one metal in the core are the same or different.

46. The method of claim 45, wherein the at least one metal on the surface and the at least one metal in the core are independently selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

47. The method of claim 46, wherein the at least one metal on the surface and the at least one metal in the core are independently selected from the group consisting of gold, silver, platinum and palladium.

48. The method of claim 47, wherein the at least one metal on the surface and the at least one metal in the core are independently gold or silver.

49. The method of claim 45, wherein the at least one metal on the surface and the at least one metal in the core are different.

50. The method of claim 49, wherein the at least one metal on the surface is silver and the at least one metal in the core is gold.

51. The method of claim 39, wherein the metallic nanoparticles are of a diameter between about 50 nm and about 100 nm.

52. The method of claim 39, wherein the bacteria is a gram positive bacteria.

53. The method of claim 39, wherein the bacteria is a gram negative bacteria.

54. A method of killing a bacterial cell, comprising contacting a surface of the bacterial cell with a bacterial cell killing effective amount of metallic nanoparticles, wherein the metallic nanoparticles have a surface comprising at least one metal and are about 100 nm or less in diameter.

55. The method of claim 54, wherein the at least one metal on the surface is selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

56. The method of claim 55, wherein the at least one metal on the surface is selected from the group consisting of gold, silver, platinum and palladium.

57. The method of claim 56, wherein the at least one metal on the surface is gold or silver.

58. The method of claim 57, wherein the at least one metal on the surface is silver.

59. The method of claim 58, wherein the surface of the metallic nanoparticles comprises silver and gold.

60. The method of claim 54, wherein the metallic nanoparticles comprise the at least one metal on the surface and at least one metal in the core, wherein the at least one metal on the surface and the at least one metal in the core are the same or different.

61. The method of claim 60, wherein the at least one metal on the surface and the at least one metal in the core are independently selected from the group consisting of gold, silver, platinum, osmium, iridium, ruthenium, rhodium, palladium, aluminum, chromium, cobalt, copper, iron, magnesium, nickel, tantalum, tin, titanium, tungsten, vanadium and zinc.

62. The method of claim 61, wherein the at least one metal on the surface and the at least one metal in the core are independently selected from the group consisting of gold, silver, platinum and palladium.

63. The method of claim 62, wherein the at least one metal on the surface and the at least one metal in the core are independently gold or silver.

64. The method of claim 60, wherein the at least one metal on the surface and the at least one metal in the core are different.

65. The method of claim 64, wherein the at least one metal on the surface is silver and the at least one metal in the core is gold.

66. The method of claim 54, wherein the metallic nanoparticles are of a diameter between about 50 nm and about 100 nm.

67. The method of claim 54, wherein the bacteria is a gram positive bacteria.

68. The method of claim 54, wherein the bacteria is a gram negative bacteria.
